(19) United States
(12) Patent Application Publication
(10) Pub. No.: US 2013/0028985 A1
(43) Pub. Date: Jan. 31, 2013

(54) COMPOSITION AND METHOD FOR TREATING MIGRAINES

(71) Applicant: Donald J. Greene, Milton, MA (US)

(72) Inventor: Donald J. Greene, Milton, MA (US)

(21) Appl. No.: 13/556,738

(22) Filed: Jul. 24, 2012

Related U.S. Application Data
(60) Provisional application No. 61/511,294, filed on Jul. 25, 2011.

(51) Int. Cl. A61K 33/06; A61P 25/06; A61P 29/00; A61K 33/34

(52) U.S. Cl. 424/638; 424/643; 424/682

(57) ABSTRACT

Disclosed are compositions for treating or preventing migraine headaches and related headaches.

### CROSS-REFERENCE TO RELATED APPLICATIONS

This application claims priority to U.S. Provisional Application Ser. No. 61/511,294, filed Jul. 25, 2011, the contents of which are incorporated herein by reference in their entirety.

### BACKGROUND OF THE INVENTION

The invention relates to compositions and methods for treating or preventing migraine headaches using supplements.

Migraine headache is a well studied disease in which neurogenic inflammation has been implicated. Neurogenic inflammation has been postulated to be involved in a variety of human disease states and conditions, including but not limited to, migraine headache, arthritis, and fibromyalgia. The basis of these conditions may be an acquired neuronal pathway that shunts neurogenic inflammatory stimuli to the cerebral vasculature, joints or muscles, respectively.

### SUMMARY OF THE INVENTION

The present invention is based, in part, upon the discovery of an effective treatment for migraine headache. The treatment includes ingesting a combination of riboflavin, a heavy metal, and a fatty acid in an amount effective to prevent or treat a migraine headache in a subject. Riboflavin is the precursor of flavin adenine dinucleotide (FAD), an electron transport chain coenzyme.

In one aspect, the invention provides an orally ingestible formulation comprising a riboflavin compound, a heavy metal, and an unsaturated fatty acid, wherein the combination of riboflavin, heavy metal, and fatty acid is effective to prevent or treat a migraine headache when the formulation is ingested by a human subject.

In some embodiments, the riboflavin compound is riboflavin, riboflavinyl glucoside, riboflavin 5′ phosphate, riboflavin 5′ adenosine diphosphate, a riboflavin acid ester, a riboflavin butyrate, a riboflavin sodium phosphate, a riboflavin 5′-phosphate, a sodium flavinadeninedinucleotide, or a flavinmononucleotide. A preferred riboflavin compound is riboflavin.

In some embodiments, the heavy metal is magnesium, copper, or zinc.

Preferably, the heavy metal is magnesium, e.g., assimilable magnesium. In some embodiments, the magnesium is provided as magnesium oxide or magnesium citrate.

In some embodiments, the heavy metal is chelated.

In some embodiments, the fatty acid is eicosapentaenoic acid (EPA), docosahexaenoic acid (DHA), or a combination of EPA and DHA. In some embodiments, the fatty acid is provided as fish oil, i.e., the EPA or DHA is derived from fish oil.

In some embodiments, the formulation is free of other B complex vitamins.

In some embodiments, the formulation is free of other nutrients.

In some embodiments, the formulation is provided in a unit dosage form.

In some embodiments, the unit dosage form comprises 50-1000 mg riboflavin, 100-750 mg riboflavin, 250-600 mg riboflavin, or 400 mg riboflavin.

In some embodiments, the unit dosage form comprises 125-1000 mg magnesium, 250-750 mg magnesium, 450-550 mg magnesium, or 500 mg magnesium.

In some embodiments, the unit dosage form comprises 120-2500 mg fish oil, 50-2000 mg fish oil, 1000-1500 mg fish oil, or 1200 mg fish oil.

In some embodiments, the formulation further includes a b-complex vitamin. The b-complex vitamin can be, e.g., thiamine (B₁), riboflavin (B.sub₂), niacin (niacinamide), pyridoxine (B₆), cyanocobalamin (B₁₂), biotin, pantothenic acid, folic acid, inositol or combinations thereof.

In one embodiment, the unit dosage form comprises 400 mg riboflavin, 500 mg magnesium and 1,200 mg fish oil.

The formulation may further include an analgesic, aspirin, paracetamol (acetaminophen), ibuprofen.

The components of the formulation can be provided as a capsule, powder, effervescent formulation, tablet, softgel tablet, solution, suspension, emulsion, or aerosol spray.

In another aspect, the invention provides a kit comprising an orally ingestible formulation that includes a riboflavin compound, a heavy metal; and an unsaturated fatty acid. The combination of riboflavin, heavy metal, and fatty acid is effective to prevent or treat a migraine headache when the formulation is ingested by a human subject.

If desired, the riboflavin, heavy metal, and fatty acid are packaged separately in the kit.

If desired, two or more of the riboflavin, heavy metal, and fatty acid are combined in the package.

Also provided by the invention is a method of treating or preventing a migraine or stress headache in a subject, the method comprising administering to the subject a therapeutically effective amount of a pharmaceutical composition that includes a riboflavin compound, a heavy metal; and an unsaturated fatty acid. The combination of riboflavin, heavy metal, and fatty acid is effective to prevent or treat a migraine headache or stress headache when the formulation is ingested by the subject.

In another aspect, the invention provides a method of reducing the levels of a pro-inflammatory mediator associated with inflammation in a subject in need thereof by administering to the subject a therapeutically effective amount of a pharmaceutical composition comprising a combination of riboflavin, heavy metal, and fatty acid and is effective to prevent or treat a migraine headache when the formulation is ingested by a human subject.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

A composition according to the invention is prepared by administering to a subject in need thereof a riboflavin composition, an unsaturated fatty acid, and a metal containing compound.

The term “composition” is used herein to include formulations which are intended for co-administration, either sequentially or simultaneously. It is generally more convenient, however, for the composition to be in a single admixture formulation. The components of the composition can be mixed prior to ingestion, e.g., within sixty seconds of ingesting, or administered separately in a time that typically does not exceed five minutes.

A preferred riboflavin compound is riboflavin, which is also known as vitamin B2. Additional compounds that can be used include riboflavinyl glucoside, riboflavin 5′ phosphate, riboflavin 5′ adenosine diphosphate, riboflavin acid esters, riboflavin butyrate, riboflavin sodium phosphate, riboflavin 5′-phosphate sodium, flavinadeninedinucleotide and flavinmononucleotide.

A preferred heavy metal is, e.g., magnesium, copper and/or zinc. Preferably, the metal is provided in a readily assimilable form. Preferably, the metal is magnesium. Preferably, the magnesium is provided as assimilable magnesium. Suitable forms of magnesium include, e.g., magnesium oxide or magnesium citrate.

The unsaturated fatty acid component can be obtained from any convenient source, including fish oil. A suitable source of the unsaturated fatty acid component is the 1200 mg Fish Oil Omega 3 softgel tablets sold by Nature's Bounty of Bohemia, N.Y.

The compositions according to the invention may be administered in any convenient form known to those skilled in the art. These forms include capsules of various types, powders, effervescent formulations, tablets, solutions, suspensions, emulsions and also aerosol sprays. The compositions may be administered orally, enterally, parenterally or transdermally using appropriate technology known to those skilled in the art.

The components of the composition can be mixed prior to ingestion by the subject or administered separately. In some embodiments, two of the three components are mixed prior to ingestion by the subject. In any event, a combination of riboflavin, heavy metal, and fatty acid is selected to provide a dose effective to prevent or treat a migraine or tension headache when the composition is ingested by a human subject.

In some embodiments, the composition is provided in a unit dosage form. In general, a “unit dosage” form is one in which the therapeutically effective amount is suitable for a single dosage. The compositions may be immediate release or controlled release, and, if controlled release, are preferably sustained release. For those compounds that are orally active, oral dosage forms are preferred, in which case the carrier is one that is suitable for oral ingestion.

If desired the composition may be provided with one or more analgesics. Suitable analgesics include, e.g., aspirin (10-2000 mg), paracetamol (acetaminophen) (10-2000 mg), ibuprofen (10-2000 mg) or any non-steroidal anti-inflammatory drug.

In other embodiments, the composition is provided with other minerals and vitamins important in the stress response, including calcium, phosphorus, iron, iodine and the water soluble vitamins vitamin B₁, vitamin B₂, vitamin B₃, vitamin B₅, vitamin B₁₂, folic acid, biotin, bioflavonoids and vitamin C. A suitable source of a B complex vitamin is NatureMade (Laguna Hills, Calif.) Super B-Complex vitamins, which includes the B vitamins thiamin, riboflavin, niacin, B-6, folic acid, biotin and pantothenic acid, as well as Ascorbic Acid, Calcium Carbonate, Thiamin Mononitrate, Cellulose Gel, Niacinamide, Riboflavin, Croscarmellose Sodium, Hydroxypropyl Methylcellulose, Dibasic Calcium Phosphate, d-Calcium Pantothenate, Magnesium Stearate, Pyridoxine Hydrochloride, Folic Acid, Polyethylene Glycol, Biotin, and Cyanocobalamin

Tryptophan may also be included. In some embodiments, the tryptophan is provided without other amino acids.

While a composition according to the invention is useful for treating or preventing a migraine or stress headache in a subject, the artisan will recognize that the composition can be used to treat additional conditions or disorders associated with pain. Pain is a heterogeneous disorder and includes all types of pain, including acute and persistent pain. In some embodiments, the composition is used to treat or prevent cluster headaches.

Additional conditions for which a composition of the invention can be used include, e.g., pain associated with jaw pain, facial pain, atypical facial pain, post-traumatic headache, cervical pain and muscle spasm, craniofacial neuralgia. neuropathic pain, diabetic neuropathy, fibromyalgia, pain associated with somatoform disorders, arthritic pain, cancer pain, neck pain, shoulder pain, back pain, cluster headaches, tension-type headache, migraine, herpes neuralgia, phantom limb pain, central pain, dental pain, NSAID-resistant pain, visceral pain, surgical pain, post-operative pain, bone injury pain, pain during labor and delivery, pain resulting from burns, post-partum pain, angina pain, genitourinary tract-related pain, neuropathic pain, diabetic neuropathy, fibromyalgia, pain associated with somatoform disorders, arthritic pain, cancer pain, neck pain, shoulder pain, back pain, cluster headaches, tension-type headache, migraine, herpes neuralgia, phantom limb pain, central pain, dental pain, NSAID-resistant pain, visceral pain, surgical pain, post-operative pain, bone injury pain, pain during labor and delivery, pain resulting from burns, including sunburn, post-partum pain, angina pain, and genitourinary tract-related pain including cystitis. In some embodiments, the composition is used to treat or prevent nociceptive pain.

The composition can be used to prevent a headache from beginning, to treat a migraine headache that has begun and/or to prevent or minimize a recurrence of a headache. Typically, the effectiveness of treatment is assessed by assessing a lessening of one or more traits associated with the condition. For example, for migraines one will evaluate one or more of maximal severity, associated vomiting, and migraine frequency. Effectiveness of treatment can also be monitored using additional methods known in the art, e.g., with the the “Migraine-Specific Quality of Life” questionnaire (M.S.Q. v.2.1), Copyright 1998 by Glaxo-Wellcome Inc., which protocol is distributed by the Medical Outcome Trust of Boston, Mass. 02116 (PMB #5031). The MSW is a 14-item questionnaire that assesses aspects of health believed to be particularly affected by migraine. Three dimensions are measured: role-function restrictive (the degree to which performance of normal activities is restricted or limited by migraine); role function preventive (the degree to which performance of normal activities is presented or interrupted by migraine); and emotional function (the emotional effects of migraine). For example, typical questions address migraine-associated problems in attending social activities (role function restrictive) or the degree to which a migraine patient feels their migraines are a burden for others (emotional function). The MSQ has shown evidence of reliability and validity in migraine suffers participating in clinical trials.

Also within the invention is the use of a vitamin and mineral combination, or of any one or two of its components when for sequential or simultaneous co-administration with the other(s), in the manufacture of a medicament to prevent or treat migraine or stress headaches, said combination providing in effective amounts assimilable magnesium, riboflavin, and fish oil.

The composition may be consumed in any form that delivers an effective dose to a subject. In one embodiment, it is provided as a dietary supplement, e.g., a capsule, a tablet, a caplet, a liquid beverage, a powder beverage mix, a dietary gel, or as a ready-to-eat bar or drink product. The dosage form of a dietary supplement may be provided in accordance with customary processing techniques for herbal and/or dietary supplements, wherein the active ingredients are suitably processed and compressed into, e.g., one or more tablets and/or caplets, with suitable excipients.

The composition may further comprise natural and/or artificial flavoring components, dyes or other coloring additives, preservatives and other conventional food supplement additives known in the art. If desired, the dietary supplement may further contain additional components to further increase the speed and or ease with which the substances enter the bloodstream. A dietary supplement form of the composition may be provided in accordance with customary processing techniques for herbal and/or dietary supplements in any of the forms mentioned above. For instance, in the embodiment in which the supplemental composition is provided is a capsule, the active ingredients may be suitably processed and encapsulated into capsules, e.g., cellulose, gelatin, etc., or compressed into caplets with cellulose, croscarmellose sodium, stearic acid, magnesium stearate, silica and other with suitable excipients. Those of skill in the art will appreciate that the supplemental composition may contain a variety of excipients. Typically, a desired daily dosage is achieved by administering an appropriate quantity of unit dosages.

The composition may alternatively be formulated for other modes of delivery, e.g., for sublingual, transnasal, parenteral, subtopical transepithelial, as a transdermal patch, subdermal, or inhalation delivery. Preparation for delivery in a transdermal patch can be performed using methods also known in the art, including those described generally in, e.g., U.S. Pat. Nos. 5,186,938; 6,183,770; 4,861,800; 4,284,444 and WO 89/09051. Patches can be made to control the release of skin-permeable active ingredients over a 12 hour, 24 hour, 3 day, and 7 day period. The penetration through skin of specific formulations may be measures by standard methods in the art (for example, Franz et al., J. Invest. Derm. 64:194-195 (1975)).

The composition may also be delivered in an aerosol spray preparation from a pressurized pack, a nebulizer or from a dry powder inhaler. Suitable propellants that can be used in a nebulizer include, for example, dichlorodifluoro-methane, trichlorofluoromethane, dichlorotetrafluoroethane and carbon dioxide. The dosage may be determined by providing a valve to deliver a regulated amount of the compound in the case of a pressurized aerosol.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral, intranasal or respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices that deliver the formulation in an appropriate manner.

Additional formulations suitable for other modes of administration include rectal capsules or suppositories. For suppositories, traditional binders and carriers may include, for example, polyalkylene glycols or triglycerides; such suppositories may be formed from mixtures containing the active ingredient in the range of 0.5% to 10%, preferably 1%-2%.

The composition can be provided as an article of manufacture in combination with labeling that the composition is to be taken for decreasing symptoms of a migraine headache or other pain as described herein. The article is typically a container or kit such as a bottle or box containing unit dosage forms (e.g., tablets, capsules) with labeling indicating the rate at which the unit dosage should be consumed.

The invention is further illustrated in the following non-limiting examples.

### EXAMPLE 1

### Composition for Treating a Migraine

An adult human subject suffers from incapacitating migraines occurring on average of once every three of four months. The headaches typically begin with a visual aura and numbness, followed by intense migraine headache pain, occasionally suffering from nausea as well. The subject is postdromal on the following day and functions at limited capacity. The subject additionally reports rebound headaches, where migraines would effectively repeat each day for up to five concurrent days.

A composition for treating the subject's migraines is prepared with a daily doses of Vitamin B2, fish oil, b complex vitamins and magnesium as follows:

B2 in four 100 mg tablets;

magnesium in a single 500 mg tablet or capsule;

a b-complex vitamin in a single tablet; and

fish oil in single capsule.

After consuming the composition, the frequency of migraines has diminished to about once every six months. The intensity has diminished dramatically as well, and the treatment has been effective for over four years in moving the subject from full incapacitation to being able to function fully, including caring for his two children and preparing them dinner. No rebound headaches were experienced after taking the combination.

Additional embodiments are within the claims.

What is claimed is:

1. An orally ingestible formulation comprising
a riboflavin compound;
a heavy metal; and
an unsaturated fatty acid,
wherein the combination of riboflavin, heavy metal, and fatty acid is effective to prevent or treat a migraine headache when said formulation is ingested by a human subject.

2. The formulation of claim 1, wherein said riboflavin compound is riboflavin, riboflavinyl glucoside, riboflavin 5′ phosphate, riboflavin 5′ adenosine diphosphate, a riboflavin acid ester, a riboflavin butyrate, a riboflavin sodium phosphate, a riboflavin 5′-phosphate, a sodium flavinadeninedinucleotide, or a flavinmononucleotide.

3. The formulation of claim 1, wherein said riboflavin compound is riboflavin.

4. The formulation of claim 1, wherein said heavy metal is magnesium, copper, or zinc.

5. The formulation of claim 1, wherein said heavy metal is magnesium.

6. The formulation of claim 5, wherein said magnesium is assimilable magnesium.

7. The formulation of claim 5, wherein said magnesium is magnesium is provided as magnesium oxide or magnesium citrate.

8. The formulation of claim 1, wherein said heavy metal is chelated.

9. The formulation of claim 1, wherein said fatty acid is eicosapentaenoic acid (EPA), docosahexaenoic acid (DHA), or a combination of EPA and DHA.

10. The formulation of claim 1, wherein said fatty acid is provided as fish oil.

11. The formulation of claim 9, wherein said EPA or DHA is derived from fish oil.

12. The formulation of claim 1, wherein said formulation is free of other B complex vitamins.

13. The formulation of claim 1, wherein said formulation is free of other nutrients.

14. The formulation of claim 1, wherein said formulation is provided in a unit dosage form.

15. The formulation of claim 14, wherein said unit dosage form comprises 50-1000 mg riboflavin.

16. The formulation of claim 14, wherein said unit dosage form comprises 100-750 mg riboflavin.

17. The formulation of claim 14, wherein said unit dosage form comprises 250-6000 mg riboflavin.

18. The formulation of claim 14, wherein said unit dosage form comprises 400 mg riboflavin.

19. The formulation of claim 14, wherein said unit dosage form comprises 125-1000 mg magnesium.

20. The formulation of claim 14, wherein said unit dosage form comprises 250-750 mg magnesium.

21. The formulation of claim 14, wherein said unit dosage form comprises 450-550 mg magnesium.

22. The formulation of claim 14, wherein said unit dosage form comprises 500 mg magnesium.

23. The formulation of claim 14, wherein said unit dosage form comprises 120-2500 mg fish oil.

24. The formulation of claim 14, wherein said unit dosage form comprises 250-2000 mg fish oil.

25. The formulation of claim 14, wherein said unit dosage form comprises 1000-1500 mg fish oil.

26. The formulation of claim 14, wherein said unit dosage form comprises 1200 mg fish oil.

27. The formulation of claim 1, further comprising a b-complex vitamin.

28. The formulation of claim 27, wherein said b-complex vitamin is thiamine (B₁), riboflavin (B.sub₂), niacin (niacinamide), pyridoxine (B₆), cyanocobalamin (B₁₂), biotin, pantothenic acid, folic acid, inositol or combinations thereof.

29. The formulation of claim 14, wherein said unit dosage form comprises
400 mg riboflavin;
500 mg magnesium;
1,200 mg fish oil.

30. The formulation of claim 1, wherein said formulation is provided as a capsule, powder, effervescent formulation, tablet, solution, suspension, emulsion, or aerosol spray.

31. The formulation of claim 1, further comprising an analgesic.

32. The formulation of claim 31, wherein said analgesic is aspirin, paracetamol (acetaminophen), or ibuprofen.

33. A kit comprising an orally ingestible formulation comprising
a riboflavin compound;
a heavy metal; and
an unsaturated fatty acid,
wherein the combination of riboflavin, heavy metal, and fatty acid is effective to prevent or treat a migraine headache when said formulation is ingested by a human subject.

34. The kit of claim 33, wherein said riboflavin, heavy metal, and fatty acid are packaged separately in said kit.

35. The kit of claim 33, wherein two or more of riboflavin, heavy metal, and fatty acid are combined in said package.

36. A method of treating or preventing a migraine or stress headache in a subject, said method comprising administering to said subject a therapeutically effective amount of a pharmaceutical composition comprising:
a riboflavin compound;
a heavy metal; and
an unsaturated fatty acid,
wherein the combination of riboflavin, heavy metal, and fatty acid is effective to prevent or treat a migraine headache or stress headache when said formulation is ingested by said subject.
